(19) United States
(12) Patent Application Publication
(10) Pub. No.: US 2025/0302760 A1
(43) Pub. Date: Oct. 2, 2025

(54) BIODEGRADABLE, CONTROLLED RELEASE MICROCAPSULES

(71) Applicant: ENCAPSYS, LLC, Appleton, WI (US)

(72) Inventors: Nianxi Yan, Appleton, WI (US); Sue M Geiger, Freedom, WI (US)

(73) Assignee: ENCAPSYS, LLC, Appleton, WI (US)

(21) Appl. No.: 19/090,712

(22) Filed: Mar. 26, 2025

Related U.S. Application Data
(60) Provisional application No. 63/570,846, filed on Mar. 28, 2024.
(60) Provisional application No. 63/652,092, filed on May 27, 2024.

(51) Int. Cl. A61K 9/50; A61K 8/11; A61L 15/42

(52) U.S. Cl. CPC A61K 9/5052; A61K 8/11; A61K 9/5089; A61L 15/42

(57) ABSTRACT

Controlled release biodegrading microcapsules comprising the reaction product of at least one protein component and a cross-linking component, the cross-linking component comprising at least one isocyanate and at least one of an acid dichloride, a chloroformate and/or a sulfonyl chloride.

### RELATED APPLICATIONS

The present application claims the benefit of prior U.S. Provisional Patent Application No. 63/570,846, filed Mar. 28, 2024, entitled “Biodegradable, Controlled Release Microcapsules” and No. 63/652,092, filed May 27, 2024, entitled “Biodegradable, Controlled Release Microcapsules”, the contents of both of which are hereby incorporated herein by reference in their entirety.

### FIELD

The present disclosure relates to biodegradable, controlled release microcapsules whose microcapsule walls comprise the reaction product of one or more protein-based components selected from proteins, peptides and/or polypeptides with at least two different cross-linking agents, at least one of which is an isocyanate component and the other of which is a chloro component.

### BACKGROUND

Microcapsules and microencapsulation technology are old and well known and their commercial applications varied. Microcapsules have played a significant role in various print technologies where a paper or other like substrate is coated with microcapsules containing ink or an ink-forming or inducing ingredient which microcapsules release the ingredient, generating an image, when fractured by pressure, as by a printing press or a stylus. Microcapsules have also played a significant role in various adhesive and sealant technologies including the encapsulation of solvents for solvent swellable/tackified preapplied adhesives whereby fracture of the microcapsules releases the solvent which softens or tackifies the adhesive to enable bonding and which re-hardened upon evaporation of the solvent. In other adhesive and sealant applications, the microcapsules contain one or more components of a curable or polymerizable adhesive or sealant composition which, upon release, leads to the cure or polymerization of the adhesive or sealant. In all of these early applications, functionality and efficacy, especially for long term storage and utility, is dependent upon the integrity of the microcapsule walls where the sought after integrity pertains to both strength, so as to avoid premature fracture, as well as impermeability, so as to prevent leakage and/or passage of the contents of the microcapsule through the microcapsule walls. In the former situation, parts having a preapplied microencapsulated adhesive have a tendency to bond together if they hit one another or are stacked upon one another where the pressure of the stack is sufficiently high to induce fracture of the microcapsules. Even if not bonded, the fracture of the microcapsules results in less adhesive to effect the bond when the bond is intended. Similarly, if the microcapsule walls allow permeation of the active components through the cell wall, even a slow permeation, the product is short lived as cure will be effected when not intended.

As with most any technology, evolution of microencapsulation technology has led to many new applications, including applications that require changes in the physical properties of the microcapsules, especially their walls. New applications require microcapsules that fracture more readily, with less pressure, but not prematurely. Other applications require microcapsules that specifically allow for a controlled, slow release or permeation of the contents from within the microcapsules without the need to actually fracture the same. For example, perfume containing microcapsules are oftentimes applied to advertising inserts in magazines so that the reader can sample the smell of the perfume. Here strength is needed to avoid premature fracturing of the microcapsules due to the weight and handling of the magazine; yet, the microcapsules need ease of fracture so that the reader can simply scratch the treated area to release the contents of the microcapsule. At the same time, it is desirable to allow for some release of the contents, even without fracturing, to induce the reader to want to scratch the sample to get a more accurate sense of the smell.

Another application for microcapsules is in laundering and fabric treatments. A number of products exist wherein microcapsules of various ingredients, including perfumes, are applied to strips of a fabric material and added to the dryer wherein the tumbling action and/or heat of the dryer causes the microcapsules to fracture, releasing the ingredients which, in a volatilized state, permeate and deposit upon the contents of the dryer. This methodology applies that “fresh out of the dryer” smell, but is short lived as the perfume continues to volatilize from the treated fabric. Other products exist whereby microcapsules containing perfumes and other ingredients are applied directly or indirectly to the fabric, especially apparel, to provide a longer-lived freshness to the same. Here, the performance or efficacy of these products is oftentimes short lived as the content of the microcapsules escapes too readily from the microcapsules and/or the walls of the microcapsules are too weak and/or have too little give such that normal wearing of the fabric causes the microcapsules to break too readily.

Whether applications have driven the evolution of microcapsule technology or the evolution of microcapsule technology has driven their expanded applications, or perhaps a little of both, there has been and continues to be constant development in microencapsulation technology, both in terms of their production/process methodology and their chemistry. Early melamine formaldehyde microcapsules continue to evolve; yet concurrently, they have, to some extent, given way to acrylic and other microcapsule chemistries and technologies. In turn, both have continued to evolve further to dual walled microcapsules of each chemistry as well as both chemistries. While the basic building blocks of the capsule walls have largely remained the same, the specific selection of building blocks and methodology has led to newer and improved microcapsules enabling the microencapsulation of a broader array of ingredients, compounds and elements.

With all the advances and improvements noted and the continued expansion of microcapsule use for a myriad of applications, there is a growing buildup of microcapsules in the environment and, in following, in animal species feeding on materials containing and/or contaminated with microcapsules. Unfortunately, these microcapsules are formed of synthetic polymeric materials and remain in the environment for decades, if not centuries. Although not yet required by various governmental/-regulatory agencies, movement is afoot to control the use of such polymeric microcapsules.

Accordingly there is a growing and, some may say, an urgent need for microcapsules whose walls are biodegradable. While capsules and, in some instances, microcapsules whose walls are formed of gelatin, albumin, polylactide and poly (lactide-co-glycolide), all generally considered biodegradable materials, have been tested, particularly for use in the pharmaceutical and nutritional supplement industries, these materials are and their characteristics make them difficult to use, particularly in the production of microcapsules. Additionally, the resultant microcapsules are lacking in their physical properties and performance as compared to traditional, generally non-biodegradable, microcapsules, Microcapsules have also been formed of block polypeptides; however, again their properties and performance are limited: certainly not appropriate for the myriad of commercial applications of traditional microcapsules. Additionally, their method of production, solvent evaporation, is not suitable for commercial large-scale production, let alone, encapsulation of the breadth of materials capable of being microencapsulated by more conventional microencapsulation techniques.

Despite the advances made, particularly from a biodegradability perspective, there remains a continuing need to balance degradability with performance profiles of the microcapsule walls. Specifically, while improved biodegradable microcapsules have been developed using select peptides and gelatins with isocyanate cross-linkers, see e.g., U.S. Pat. No. 11,952,492 B2 and US Patent Application Publication 2023/0112578A1, there is still a need to address shell integrity and performance, as well as a desire to be able to control or dial-in specific performance profiles to facilitate the production of unique/custom microcapsules for specific end-use applications.

In following, it has now been found that one is able to produce further improved biodegradable microcapsules and/or better control the resulting physical properties and release characteristics of such biodegradable microcapsules through the concurrent use of multiple cross-linking agents of different chemistries.

### SUMMARY

According to the present teachings there are provided microcapsules and methods for the production thereof wherein the microcapsules comprise the reaction product of A) one or more protein-based components selected from proteins, peptides and/or polypeptides, and B) a cross-linking component comprising a combination of two or more cross-linking agents, at least one of which is i) an isocyanate component selected from the group consisting of one or more diisocyanates and/or poly-isocyanates and at least one of which is ii) a chloro component selected from the group consisting of a) di- and/or poly-acid chlorides, b) bis- and/or poly-chloroformates and/or c) di- and/or poly-sulfonyl chlorides; wherein (I) the weight ratio of protein-based components to the cross-linking component (A:B) is from 100:1 to 1:100, preferably from 50:1 to 1:50, more preferably from 10:1 to 1:10, especially from 1:5 to 1:0.2, most preferably from 1:2 to 1:1 and (II) the weight ratio of the isocyanate component to the chloro component (iii) is from 100:1 to 100:50, preferably from 100:2.5 to 100:25, more preferably from 100:5 to 100:15, most preferably from 100:7.5 to 100:12.5.

The microencapsulation process of the present teaching is employed to provide carrier microcapsules containing various core materials including solids, hydrophilic agents, hydrophobic agents, lipophilic agents and the like, especially UV absorbers, UV reflectors, pigments, dyes, colorants, scale inhibitors, corrosion inhibitors, antioxidants, pour point depressants, waxes, deposition inhibitors, dispersants, flame retardants, biocides, active dye tracer materials, odor control agents, natural oils, flavor and perfume oils, crop protection agents, pharmaceuticals, medicaments, phase change materials and the like.

### DETAILED DESCRIPTION

The present teaching is directed to novel microcapsules and the process by which they are made, In particular, the present teaching is directed to microcapsules that are biodegradable and serve as carriers for various core materials contained therein including solids, hydrophilic agents, hydrophobic agents, lipophilic agents and the like. Most especially, the present teaching is directed to carrier microcapsules that are biodegradable and have controlled release properties for the liquid/volatile core materials contained therein and/or have improved or customized physical properties, such as strength, fracturability, and the like, for their intended purpose.

According to the present teachings there are provided microcapsules and methods for the production thereof wherein the microcapsules comprise the reaction product of A) one or more protein-based components selected from proteins, peptides and/or polypeptides, and B) a cross-linking component comprising a combination of two or more cross-linking agents, at least one of which is i) an isocyanate component selected from the group consisting of one or more diisocyanates and/or poly-isocyanates and at least one of which is a ii) chloro component selected from the group consisting of a) di- and/or poly-acid chlorides, b) bis- and/or poly-chloroformates and/or c) di- and/or poly-sulfonyl chlorides; wherein (I) the weight ratio of protein-based components to the cross-linking component (A:B) is from 100:1 to 1:100, preferably from 50:1 to 1:50, more preferably from 10:1 to 1:10, especially from 1:5 to 1:0.2, most preferably from 1:2 to 1:1 and (II) the weight ratio of the isocyanate component to the chloro component (iii) is from 100:1 to 100:50, preferably from 100:2.5 to 100:25, more preferably from 100:5 to 100:15, most preferably from 100:7.5 to 100:12.5.

Similarly, the process by which the present microcapsules are prepared is unique in that it calls for the use of both the protein-based components and the combination of the two or more crosslinking agents as the wall forming materials. The microencapsulation process may be a water-in-oil process, an oil-in-water process or a water-in-oil-in water process: the latter process is especially useful when the core material is hydrophilic or water soluble/dispersible and one wants to avoid the use of large volumes of oil phase materials as is required of the water-in-oil process. For convenience, the following description is presented in terms of the oil-in-water process: though those skilled in the art will readily appreciate and acknowledge the adaptability of the process to a water-in-oil and water-in-oil-in-water process.

The first critical component for use in the production of the microcapsules of the present teaching is the protein-based component. As noted, this component is selected from proteins, peptide and polypeptides as well as combinations thereof. A common factor amongst all of these is they have, or at least a majority of such component, preferably at least 70 mole percent, most preferably at least 80 mole percent, most preferably at least 90 mole percent, must have at least two bonding sites for reacting with the cross-linkers. The protein-based component is preferably a natural protein component, though synthetic equivalents may be used as well. Both plant and animal proteins may be used; though preferably, the protein component is derived from plants. Exemplary animal protein-based components include animal proteins, animal polypeptides, animal peptides, egg protein, fibroin, dairy protein, gelatin and combinations of one more of the foregoing, especially gelatin. Exemplary plant proteins include, but are not limited to, soy protein, pea protein, chickpea protein, beans protein, lentils protein, potato protein, wheat protein, oats protein, malt protein, rye protein, barley protein, rice protein, algae protein, gluten protein, lupin protein, other legume proteins, and mixtures thereof. Whatever the source of the protein-based component, they may be used or sourced in the form of concentrates, isolates, or a combination thereof. Such protein-based materials are widely available commercially and include food or pharmaceutical grade and non-food grades depending upon the end-use application for the resulting microcapsules. Given the lack of a clear demarcation as between proteins and polypeptides and between peptides and polypeptides, the discussion herein will refer to proteins and polypeptides: though all three are understood to be encompassed thereby, particularly as context allows.

Generally speaking, suitable proteins are those of from 5,000 Da to 110,000 Da, preferably from 10,000 Da to 80,000 Da, more preferably 10,000 Da to 65,000 Da, most preferably from about 15,000 Da to about 40,000 Da. Although proteins on the higher end, up to 110,000 Da, can be used, caution must be employed as they, particularly the gelatins, tend to be tacky, particularly in the presence of moisture, which can adversely affect the intended end-use application. Of course, one can adjust properties by employing two or more proteins having different weight average molecular weights; however, in such instances it is preferred that the overall weight average molecular weight of the combination is adjusted to fall within the preferred numerical ranges described above.

Especially preferred animal proteins are gelatins, which are well known and widely available and are broadly used across a number of industries for encapsulation of, e.g., food substances, pharmaceuticals, cosmetics, agricultural products and the like. Gelatin is typically prepared either by partial acid hydrolysis (gelatin type A) or alkaline hydrolysis (gelatin type B) of native collagen that is found in animal collagen from skins, cartilage, bones, and tendons, especially those of fish, cattle, swine, chickens, and the like. The surface of gelatin is negatively charged at higher pH and positively charged at lower pH (pH 5). The isoelectric point of gelatin A is in the region of 8-9, while it is about pH 4.8 to 5.4 for gelatin type B. Gelatin for use in the present teaching also includes gelatin hydrolysates prepared by hydrolysis of gelatin with a protease such as collagenase and cysteine protease. Suitable gelatins will typically have a Bloom value of from 55 to 325. Typically, the weight average molecular weight of the gelatin is determined in accordance with the methods specified in “20-1 Molecular weight distribution” and “20-2 Average molecular weight” in “PAGI METHOD, Tenth edition” (Commission on Testing Method for Photographic Gelatin, 2006). Preferred gelatins are those which have undergone an acid treatment as such gelatins often facilitate or enable better control on particle size, particularly if one is seeking to control particle size to a single-digit micrometer order. Similarly, from the perspective of gel network formation, it is preferred to use a gelatin having a relatively low jelly strength. Specifically, it is preferred to use a gelatin which exhibits a jelly strength according to JIS K6503-2001 of 10 to 200.

Especially preferred plant proteins are those or their isolates/fractions that are readily soluble in water (or the aqueous phase) or nearly so, at least at the levels or concentrations to be used in producing the microcapsules of the present teaching. The present teaching is also applicable to those plant proteins and/or isolates/fractions that are insoluble or poorly soluble and which have been subjected to fragmentation to make them more soluble. Again, preferred plant proteins include, but are not limited to, soy protein, pea protein, chickpea protein, beans protein, lentils protein, potato protein, wheat protein, oats protein, malt protein, rye protein, barley protein, rice protein, algae protein, gluten protein, lupin protein, other legume proteins, and mixtures thereof. These proteins may be in the form of protein concentrates, protein isolates, or a combination thereof, with or without fragmentation. Such proteins are widely available commercially and include food or pharmaceutical grade and non-food grades depending upon the end-use application for the resulting microcapsules.

As noted above, although the proteins may be used as is, it is often desirable to employ proteins which have been subjected to fragmentation whereby protein compositions comprising protein segments of various lengths, though of overall lesser median length, and make-up are prepared. This is particularly so in the case of proteins, especially plant proteins, that are insoluble or poorly soluble in water. Nevertheless, any of the known protein extracts and/or isolates may be subjected to the fragmentation process, regardless of their water solubility: though it is especially beneficial for those which are water insoluble or poorly soluble. In this regard, even proteins that are not poorly soluble, i.e., those of less than 100% soluble but more than poorly soluble, can benefit from fragmentation, improving overall solubility of the protein. For the purpose of this disclosure, proteins are considered insoluble if their solubility in water at room temperature and neutral pH is less than 25%, poorly soluble proteins will generally have a solubility at those conditions of from 25% to less than 70%. Again, while it is recognized that certain protein isolates or fractions have varying solubility, particularly under different conditions; all such isolates and fractions may benefit from fragmentation, especially those fractions whose solubility parameters fall within the foregoing ranges as well. In following, reference herein to fragmented proteins includes fragmented extracts as well as fragmented isolates: the latter having already undergone some fractionation.

The use of fragmentated proteins, oftentimes allows one to better control and/or manipulate the final properties and performance characteristics of the resulting microcapsules. As note above, fragmentation is especially useful with proteins that are insoluble and/or difficult to disperse in the reaction phase to which they are added due to their size and/or physical conformation, i.e., folded or unfolded: enabling and/or facilitating, respectively, their use in microencapsulation processes. Fragmentation of the proteins may be achieved by any of the known methods for breaking protein chains, with or without first unfolding the chains. Such methodologies include mechanical shear such as homogenization, ultrasonication, pulverization and the like; pH degradation; chemical degradation such as glycation, phosphorylation, acylation, etc.; high temperature degradation; solvent degradation; salt degradation; and combinations thereof. Exemplary fragmentation methods are further described in Grossman, L., et., “Current insights into protein solubility: A review of its importance for alternative proteins,” Food Hydrocolloids, 137 (2023) 108416, the contents of which are incorporated herein by reference. Preferably, the fragmentation process involves several of the foregoing parameters, more preferably one in which one of the parameters is a mechanical shear. Most preferably, the fragmentation process involves a mechanical shear together with pH and/or temperature control/modification for a specified period time or duration of fragmentation. Generally speaking, the resulting fragmented protein isolates typically comprise protein fragment compositions having a median particle size, i.e., D50, which is at least about 40% less, more preferably at least about 50% less, most preferably at least about 60% less, than the median particle size of the protein isolate prior to fragmentation. Preferred fragmented proteins have a median particle size that is from about 0.1% to about 60%, more preferably from about 5% to about 35%, of the median particle size of the plant protein isolate prior to fragmentation. The ultimate particle size and distribution thereof is both a factor of the fragmentation process use and its duration as well as the desired properties of the resulting microcapsules.

Suitable peptides for use in the practice of the present teaching are polypeptides having at least two sites reactive with the cross-linking agents and having an average molecular weight (Da) of less than 10,000, preferably 7,000 or less, most preferably 5500 or less, generally from 2, preferably 5, to 100 amino acids. Preferred polypeptides comprise linear chains of from 5 to 50, preferably 15-45, most preferably 20-40 amino acids, typically, though not exclusively, bonded to one another through a peptide bond. Other bonds may be present in the linear chain including disulfide bonds, sulfur links, urea bonds and the like: though such other bonds will be few in number per peptide chain, typically less than 20%, preferably less than 10%, of the bonds in the peptide chain. As noted, the reactive sites on the peptides are preferably primary amino reactive sites, though secondary amino and hydroxyl groups may also be present: most preferably all or significantly all of the reactive sites are primary amino reactive sites.

As noted above, the peptides may be derived from natural sources or they may be synthetic, especially those formed by the solid phase peptide synthesis (SPPS) of amino acids. While the most common amino acids have the amine group on the alpha carbon atom along with the carboxyl moiety, the present teaching is not limited to peptides of those amino acids and it is understood that those peptides formed of amino acids wherein the amino group is on the beta or gamma carbon atom and, to a lesser extent, the delta carbon atom are also contemplated and desirable. Most especially the peptides are formed of the alpha and beta amino acids and combinations thereof, most especially the alpha amino acids. Additionally, it is to be appreciated that the amino acids are not limited to those that have a single amine and a single carboxyl group. Rather, suitable amino acids also have a plurality of either or both, e.g., two amine groups and/or two carboxyl groups, as well as other reactive moieties/sites including, for example, the presence of sulfur containing groups or moieties in or on their side chain: the latter providing disulfide links or sulfur bridges. Amino acids are well known, numbering in the hundreds, though the present teaching is especially directed to, but not limited to, peptides formed of the more common and abundant amino acids, as described in greater detail below and as will be appreciated by those skilled in the art.

While the natural peptides are, for the most part, derived from proteinogenic amino acids (they may also contain low levels of non-proteinogenic amino acids), synthetic peptides may be derived from proteinogenic amino acids, non-proteinogenic amino acids and combinations of both, especially, for example, those peptides comprising both the L- and D-isomers of the same or different amino acids. Proteinogenic amino acids are the L-α-amino acids including the L isomers of arginine, histidine, lysine, aspartic acid, glutamic acid, serine, threonine, asparagine, glutamine, cysteine, selenocysteine, glycine, proline, alanine, isoleucine, leucine, methionine, phenylalanine, tryptophan, tyrosine, valine and pyrrolysine. Non-proteinogenic amino acids include the D isomers of the foregoing as well as, for example, but not limited to, α-amino butyric acid, norvaline, norleucine, alloisoleucine, t-leucine, α-amino-n-heptanoic acid, pipecolic acid, α, β-diaminoproprionic acid, α, γ-diaminobutyric acid, omithine, allothreonine, β-alanine, β-aminobutyric acid, α-aminoisobutryric acid, isovaline, sarcosine, N-ethylglycine, N-propylglycine, N-isopropylglycine, N-methylalanine, N-ethylalanine, N-methyl-β-alanine, N-ethyl-β-alanine, isoserine, α-hydroxy-γ-aminobutyric acid, ornithine, phenylalanine, β-aminobutyric acid, and the like. Of course, the peptides may also be formed of the salts of the amino acids, especially the monohydrochloride salts, including, for example, D-lysine monohydrochloride, L-lysine monohydrochloride, L-arginine monohydrochloride, D-arginine monohydrochloride, L-orthinine monohydrochloride, D-monohydrochloride, and the like.

The peptides used in the practice of the present teaching may also be formed of lower peptides, generally those having 10 or fewer amino acids, preferably 2-5 amino acids, especially dipeptides, tripeptides, and tetrapeptides. Exemplary lower peptides include, but are not limited to the doublet amino acids, such as dialanine, dilysine, diglycine, etc. as well as dipeptides that have other than a peptide bond, including, for example, cystine, cystathionine, ianothianine, djenkolic acid, and diaminopimelic acid, among others.

The peptides may be homopeptides, wherein the peptide is comprised of the same amino acid, and copeptides, where the peptide is comprised of two or more different amino acids and/or lower peptides, as well as combinations of the two. The copeptides include those peptides wherein the peptide comprises both the L- and D-isomers of the same amino acid. Copeptides also include those peptides having a plurality of amino acids and/or doublet amino acids and/or lower peptides randomly linked along the peptide chain or in a structured arrangement, i.e., block peptides, wherein blocks of low to moderate molecular weight peptides, which may be homopeptides or copeptides, are bonded to one another to make up the full peptide. Such block peptides may be random copeptides, e.g. A-B-A-C-B-A or structured, e.g., A-A-A-C-C-C-B-B-A-A-A, the latter being formed by known techniques such as the sequential polymerization of the low to moderate molecular weight peptide blocks, wherein A, B and C represent low to moderate molecular weight peptides, i.e, oligopeptides of 2 to 30 amino acids, preferably 3-20 amino acids, more preferably 5 to 10 amino acids.

Also contemplated are peptides wherein one or both ends of the peptide chain are terminated with a select amino acid. For example, it may be desirable to have amine or hydroxyl functionality at or near both ends of the peptide chain. The former allowing for polyurea microcapsule wall formation and the latter for polyurethane microcapsule wall formation.

As noted the protein-based component may be a single protein, polypeptide or peptide; a combination of proteins (including fractured proteins); a combination of polypeptides; a combination of peptides; as well as combinations of any of the foregoing. The specific protein-based component or mixture thereof chosen for any given microencapsulation depends upon a number of factors including the desired physical properties, release properties, the particular microencapsulation process to be employed, which phase the protein-based component is to be introduced into, etc. The selection and/or the make-up thereof, e.g., amino acids and their numbers, play an important role in the physical properties and release properties of the microcapsule walls. Similarly, the selection of protein-based components and their percentage make-up of the protein-based component also greatly affects their solubility/dispersibility in the phase in which it is introduced into the process as well as its affinity or aversion to the other phase. In this respect, it is well appreciated that certain proteins, polypeptides, and peptides, particularly the amino acid building blocks thereof, have polar and/or electrically charged side chains, hydrophobic side chains and the like. Hence, by selection of specific proteins, polypeptides, and peptides, and their combinations, particularly the amino acid components thereof and their concentrations, one can tailor the proteins, polypeptides and peptides for their specific need and process. Of course, one may also employ surfactants and other solubilizing/dispersing aids to assist with the solubilization and/or dispersion of the peptides in the select phase. Suitable surfactants and solubilizing/-dispersing aids include polyvinyl alcohol (PVA), polystyrene sulfonate (PSS), carboxymethylcellulose (CMC), sodium salt of naphthalene sulfonate condensate, and the like, as well as mixtures thereof.

Although the foregoing discussion has been made with respect to protein components having at least two reactive sites for reaction with the cross-linkers, it is also contemplated that such proteins having a single reactive site may be used provided that the amount of such protein is not more than about 30 mole percent, preferably no more than about 15 mole percent, more preferably no more than about 5 mole percent, of the protein component. In the event the cross-linkers include monofunctional cross-linkers, it is preferably that no or essentially no proteins be used having a single reactive site.

The second critical component for the formation of the microcapsules of the present teaching is the cross-linking component which comprises a combination of two or more cross-linking agents, at least one of which is i) an isocyanate component selected from the group consisting of one or more diisocyanates and/or poly-isocyanates and at least one of which is ii) a chloro component selected from the group consisting of a) di- and/or poly-acid chlorides, b) bis- and/or poly-chloroformates and/or c) di- and/or poly-sulfonyl chlorides.

Suitable diisocyanate and polyisocyanates for use in the practice of the present teaching include those known for use in the formation of polyurethane, polyurea, and polyurethane-urea microcapsules and are well known to those of ordinary skill in the art. These isocyanates include aliphatic, cycloaliphatic, aromatic, polyaromatic, etc., isocyanates as well as combinations thereof, and may be present as adducts, trimers, biurets, symmetric trimers, asymmetric trimers, oligomers, prepolymers and the like. Such isocyanates typically have from 2 to 16, preferably 4 to 10 carbon atoms in the basic hydrocarbon skeleton. While the diisocyanates are preferred, polyisocyanates, especially those having 3 or 4 cyanato (NCO) groups, 3 to 10 cyanato groups in the case of dimers, biurets, trimers, oligomers and prepolymers, as well as combinations of diisocyanates and polyisocyanates are also desirable and useful. While a single isocyanate is suitable, it is also desirable to employ combinations of isocyanates, e.g., a combination of di- and/or poly-isocyanates, a combination of aliphatic and aromatic isocyanates, as well as combinations of both. In this regard, the weight percent of each isocyanate may be from 0-100% of the combination. In the case of combinations of aliphatic and aromatic isocyanates, it is preferred that each be present in an amount of at least 5% by weight, preferably at least 10% by weight, more preferably at least 20% by weight, depending upon the desired leakage rate. For example, aliphatic/aromatic isocyanate microcapsules with lower leakage will typically have at least 50% by weight, more preferably at least 70% by weight of the aliphatic isocyanate. Further, while mono-isocyanates may be present, they are not required and, if present, they will comprise no more than 50 mole percent, preferably no more than 30 mole percent, more preferably no more than 20 mole percent, most preferably no more than 10 mole percent of the isocyanate component. In the event the protein-based component includes mono-functional proteins, polypeptides and/or peptides, it is especially preferred, if not critical, that no mono-isocyanates be used.

Exemplary aliphatic and cycloaliphatic isocyanates include 2,2,4-trimethylhexamethylene diisocyanate, 2,4,4-trimethylhexamethylene diisocyante, 1,4-tetramethylene diisocyanate, 1,6-hexamethylene diisocyanate (HDI), isophorone diisocyanate IPDI), 4,4′-methylene-bis-(cyclohexane diisocyanate), cylcohexane-1,4-diisocyante, and adducts, trimers, biurets, symmetric trimers, asymmetric trimers thereof, especially of hexamethylene diisocyanate, and the like, including trimethyol propane adducts of hexamethylene diisocyanate.

Aromatic isocyanates include m- and p-tetramethylxylene diisocyanate (TMXDI), α,α′-xylylene diisocyanate, methylene diisocyanate (MDI), especially 4,4′-diphenylmethane diisocyanate, toluene diisocyanate (TDI), 1,4-phenylene diisocyanate, 1,3-phenylene diisocyanate, 2,4,6-triisocyanate toluene, 4,4′,4′-triisocyanate triphenyl methane, 1,2,4-benzene triisocyanate, 2,5-norbornene diisocyanate, 3,3′-dimethyldiphenyl-4,4′-diisocyanate, naphthalene diisocyanate, and adducts, trimers, biurets, symmetric trimers, asymmetric trimers thereof, especially of toluene diisocyanate and methylene diisocyanate, and the like. Exemplary trimers include, but are not limited to, the trimer of hexamethylene diisocyante sold under the trademark Desmodur.RTM.N-3390 by Bayer Corporation of Pittsburgh, Pa. and the trimer of isophorone diisocyanate.

Suitable isocyanates also include oligomeric and low molecular weight polymeric isocyanates, i.e., prepolymers: again, these materials that are well known to those of ordinary skill in the art. Preferably, in keeping with the desirability of degradability, such oligomeric and low molecular weight polymeric isocyanates are formed of an isocyanate and low molecular weight polyols, preferably C₁ to C₆ polyols, especially diols and/or triols, most especially linear diols and triols. Alternatively, or in additional thereto, consistent with the teaching of the present specification, such oligomeric and low molecular weight polymeric isocyanates may be prepared by the reaction of individual amino acids or lower peptides, 2 to 5 amino acids with isocyanates. In either instance, in the preparation of such oligomers and low molecular weight polymers, an excess of the isocyanate is employed to provide oligomers and low molecular weight polymers with at least two free cyanate groups. Exemplary oligomeric and low molecular weight polymeric isocyanates include, but are not limited to, trimethylol propane adducts of the isocyanates, especially those of toluene diisocyanate, methylene diisocyanate, and xylylene diisocyanate.

In addition to the at least one isocyanate cross-linking agent, the microcapsules of the present teaching also include as a cross-linking agent at least one chloro-component selected from the group consisting of a) di- and/or poly-acid chlorides, b) bis- and/or poly-chloroformates and/or c) di- and/or poly-sulfonyl chlorides. In this regard, the chloro-component may be a single cross-linking agent or a mixture of such cross-linking agents: the latter allowing for two or more cross-linking agents from the same family, i.e., (a) or (b) or (c), or one or more cross-linking agents from one family with one or more cross-linking agents from a second or from all three families (a), (b) and (c).

Suitable acid chlorides generally have the formula R¹(C(O)Cl)ₓ wherein R¹ is a hydrocarbyl group of from 3 to 12, preferably from 3 to 10, more preferably 4 to 8, carbon atoms and x is 2 to 4. The hydrocarbyl groups include aliphatic and aromatic hydrocarbyl groups. The former may be saturated or unsaturated (including polyunsaturated), straight chain or branched chain hydrocarbyl groups. The latter may include substituted aromatic groups, especially those substituted with one or more low carbon number hydrocarbons in addition to the acyl chloride moiety. Most preferably the acid chlorides are derived from carboxylic acids, especially dicarboxylic acids, most especially saturated dicarboxylic acids. Exemplary acid chlorides include adipoyl chloride, octanedioyl dichloride, succinyl chloride, azelaoyl chloride, sebacoyl chloride, dodecanedioyl dichloride, isophthaloyl chloride, terephthaloyl chloride, 1,3,5-benzenetricarbonyl chloride, and 1,2,4,5-benzenetetracarbonyl tetrachloride,

Suitable chloroformates include diglycolyl chloride and those compounds generally having the formula R²(OC(O)Cl)₂ wherein R² is a saturated, straight chain or branched, hydrocarbyl group of from 2 to 10, preferably from 2 to 6, more preferably 2 to 4, carbon atoms or an ether group of formula —R³(OR³)_{y}— wherein R³ is a saturated, straight chain hydrocarbon of 1 to 6, preferably 2 to 4, carbon atoms and y is from 1 to 8, preferably from 1 to 6, more preferably from 2 to 4. Most preferably the chloroformates are derived from diols and polyether glycols, especially the diols and polyethylene glycols. Exemplary chloroformates include ethylene bis(chloroformate), 1,4-butanediol bis(chloroformate), 1,6-hexanediol bis(chloroformate), ethylene glycol bis(chloroformate), tri(ethylene glycol) bis(chloroformate), diethylene glycol bis-chloroformate, neopentyldiol bis(chloroformate). diglycolyl chloride, 1,4-phenylene bis(chloroformate), bisphenol A bis(chloroformate), and bisphenol Z bis(chloroformate),

Suitable sulfonyl chlorides generally correspond to those compounds having the formula R⁴(SO₂Cl)_{z} wherein R4 is benzyl, biphenyl, naphthyl, diphenyl ether, or methylene diphenyl moiety and z is 2 to 4, preferably 2 to 3. In those instances where there are multiple aromatic rings, fused or linked, each ring preferably has at least one sulfonyl chloride substituent. Exemplary sulfonyl chlorides include 1,3-benzenedisulfonyl chloride, 1,4-benzenedisulfonyl chloride, 1,3,5-benzenetrisulfonyl chloride, 4,4′-biphenyldisulfonyl chloride, 4,4′-methylene bis(benzenesulfonyl chloride), 4,4′-bis(chlorosulfonyl)diphenyl ether, 1,5-naphthalene disulfonyl chloride, 4,4-naphthalenedisulfonyl chloride and 2,7-naphthalenedisulfonyl chloride.

Further, while mono-functional acid chlorides, chloroformates and sulfonyl chlorides may be present, they are not required and, if present, they will comprise no more than 50 mole percent, preferably no more than 30 mole percent, more preferably no more than 20 mole percent, most preferably no more than 10 mole percent of the isocyanate component. If the protein-based component includes mono-functional proteins, polypeptides and/or peptides, it is especially preferred, if not critical, that no mono-functional acid chlorides, chloroformates and/or sulfonyl chlorides be used. Similarly, it is preferred that mono-isocyanates and mono-functional acid chlorides, chloroformates and/or sulfonyl chlorides not be used together.

In preparing the microcapsules according to the present teaching the weight ratio of the protein-based component (A) to the cross-linking component (B) is from 100:1 to 1:100, preferably from 50:1 to 1:50, more preferably from 10:1 to 1:10, especially from 1:5 to 1:0.2, most preferably from 1:2 to 1:1, (A:B). Additionally, the weight ratio of the isocyanate component (B) (i) to the chloro component (B) (ii) is from 100:1 to 100:50, preferably from 100:2.5 to 100:25, more preferably from 100:5 to 100:15, most preferably from 100:7.5 to 100:12.5 ((B)(i):(B)(ii)).

Although it is known to prepare hollow, empty microcapsules, the present teaching is especially directed to the preparations of microcapsules containing various core materials to their intended used. Core materials that may be encapsulated in accordance with the present teaching include a myriad of substances, consistent with those materials that are encapsulated by existing technologies and chemistries. Core materials include solid particles, semi-solid materials, hydrophilic liquids, lipophilic liquids, hydrophobic liquids, volatile liquids, and the like. Specific selection depends upon the intended utility of the microcapsules. Indeed, microcapsules have a myriad of applications across various industries and consumer products including, but not limited to, agrochemicals, pharmaceuticals, cosmetics industry, personal care products, laundering detergents, homecare & cleaning products, oral care, dental care, textiles, paper, mining, oil industry, water treatment, adhesives, coatings, coatings, plastics, sealants, construction, paints, inks and dye formulations. Exemplary core materials include, but are not limited to UV reflectors, UV absorbers, pigments, dyes, colorants, scale inhibitors, emollient oils, insecticides, detergents, printing inks, corrosion and rust, recording materials, inhibitors, antioxidants, pour point depressants, catalysts, initiators, waxes, deposition inhibitors, dispersants, flame retardants, biocides, active dye tracer materials, silicone conditioners, shampoos, biocides, adhesives, anti-fouling agents, odor control agents, cosmetic additives, oxidizing agents, personal care actives, agrochemicals, fertilizers, fats, nutrients, enzymes, liquid crystals, natural oils, fragrances, flavor and perfume oils, crop protection agents, medicaments, pharmaceuticals, phase change materials and the like. Specific examples of core materials are disclosed in, e.g., US 2013/0337023, U.S. Pat. Nos. 10,456,766, 8,119,214, 9,714,397, 10,485,739, 4,977,060, 10,675,277, 20,070,138673, and 20,130,302392, all of which are hereby incorporated by reference, among a myriad of other patents, patent publications and the like.

The following presents a non-limiting list of exemplary core materials.

Linear or branched hydrocarbons of different chain lengths and viscosities such as mineral oil, petrolatum, white oil (also known as paraffin oil), dodecane, isododecane, squalane, hydrogenated polyisobutylene, polybutene, polydecene, docosane, hexadecane, isohexadecane and other isoparaffins, which are branched hydrocarbons.

Alcohol, diol, triol or polyol esters of carboxylic or dicarboxylic acids, of either natural or synthetic origin having straight chain, branched chain and aryl carboxylic acids include diisopropyl sebacate, diisopropyl adipate, isopropyl myristate, isopropyl palmitate, myristyl propionate, cetyl lactate, myristyl lacate, lauryl lactate, C12-15 alkyl lactate, dioctyl malate, decyl oleate, isodecyl oleate, ethylene glycol distearate, ethylhexyl palmitate (octyl palmitate), isodecyl neopentanoate, tridecyl neopentanoate, castoryl maleate, isostearyl neopentanoate, di-2-ethylhexyl maleate, cetyl palmitate, myristyl myristate, stearyl stearate, cetyl stearate, isocetyl stearate, dioctyl maleate, octyl dodecyl stearate, isocetyl stearoyl stearate, octyldodecyl stearoyl stearate dioctyl sebacate, diisopropyl adipate, cetyl octanoate, glyceryl dilaurate, diisopropyl dilinoleate and caprylic/capric triglyceride. Naturally occurring includes triglycerides, diglycerides, monoglycerides, long chain wax esters and blends of these. Examples of naturally derived ester-based oils and waxes include, but are not limited to, argan oil, com oil, castor oil, coconut oil, cottonseed oil, menhaden oil, avocado oil, beeswax, carnauba wax, cocoa butter, palm kernel oil, palm oil, peanut oil, shea butter, jojoba oil, soybean oil, rapeseed oil, linseed oil, rice bran oil, pine oil, sesame oil, sunflower seed oil and safflower oil. Also useful are hydrogenated, ethoxylated, propoxylated and maleated derivatives of these materials, e.g. hydrogenated safflower oil, hydrogenated castor oil. Cholesterol and its esters and derivatives, as well as natural materials comprising cholesterol derivatives such as lanolin and lanolin oil.

Phospholipids (e.g. lecithin), sphingophospholipids, ceramides and related materials.

C4-C20 alkyl ethers of polypropylene glycols, C1-C20 carboxylic acid esters of polypropylene glycols, and di-C8-C30 alkyl ethers. Also included are PPG-14 butyl ether, PPG-15 stearyl ether, diodyl ether, dodecyl octyl ether, and mixtures thereof.

Saturated and unsaturated fatty acids including but not limited to oleic, palmitic, isostearic, stearic, ricinoleic, linoleic and linolenic acid. Carboxylic monoesters and polyesters of sugars (mono-, di-and polysaccharides) and related materials.

Silicones such as polyalkylsiloxanes, polydialkylsiloxanes, polydiaryisiloxanes, and polyalkarylsiloxanes may also be used. This includes the polydimethylsiloxanes, which are commonly known as dimethicones. Further cyclic siloxanes (e.g., cyclopentasiloxane) and dimethiconoles, alkyl methicones, alkyl dimethicones, dimethicone copolyols, amino-functional silicones (e.g., amodimethicone, trimethylsilyloxyamodimethicone) and amphoteric silicones (e.g., cetyl PEG/PPG-15/15 butyl ether dimethicone, and bis-PEG-18 methyl ether dimethyl silane).

Oily and oil-soluble extracts of plant materials such as flowers and herbs. This comprises a wide range of materials, with some non-limiting examples including extracts of rosemary, green, white or black tea, orchid, grape seed, sage, soybean, echinacea, arnica, rosehip, olive, artichoke. Further plant-extracted oil-soluble components such as lycopene and other mixed carotenoids, capsaicin and capsaicinoids, polyphenols (e.g., rosmarinic acid), terpenes and terpenoids, oleoresins.

Exemplary dyes include, but are not limited to, Green 6 (CI 61570), Red 17 (CI 26100), Violet 2 (CI 60725) and Yellow 11 (CI 47000). Examples of oil-dispersible pigments include, but are not limited to Beta Carotene (CI 40800), Chromium Hydroxide Green (CI 77289), Chromium Oxide Green (CI 77288), Ferric Ferrocyanide (CI 77510), Iron Oxides (CI 77491, 77492 77499), Pigment Blue 15 (CI74160), Pigment Green 7 (CI 74260), Pigment Red 5 (CI 12490), Red 30 (CI 73360), Titanium Dioxide (CI 77891) and Ultramarines (CI 77007).

Exemplary pharmaceutical active, especially for dermatological treatment of conditions of skin, hair and nails include, but is not limited to, topical anaesthetics, anti-fungal, anti-bacterial, anti-viral, anti-dandruff, anti-acne and anti-inflammatory agents (steroidal and non-steroidal).

Examples of vitamin and derivatives include tocopherol, tocopheryl acetate, retinol, retinyl palmitate, ascorbyl palmitate, niacinamide, beta carotene.

Fragrances suitable for use in accordance with the present teaching include, without limitation, any combination of perfumes, flavors, essential oils, sensates and plant extract or mixture thereof that is capable of being encapsulated in accordance with the present application. A list of suitable fragrances is provided in U.S. Pat. Nos. 4,534,891, 5,112,688, 5,145,842, 6,194,375, 20110020416 and PCT application Nos. WO2009153695 and WO2010/044834 and Perfumes Cosmetics and Soaps, Second Edition, edited by W. A. Poucher, 1959. Each of the foregoing documents is incorporated herein by reference in its entirety.

Typical representative perfume and sensate components include, but are not limited to, linalool, coumarin, geraniol, citral, limonene, citronellol, eugenol, cinnamal, cinnamyl alcohol, benzyl salicylate, menthol, menthyl lactate, eucalyptol, thymol, methyl salicylate, methylfuran, menthone, cinnamaldehyde.

Typical representative examples for essential oils include, but are not limited to, orange, lavender, peppermint, lemon, pine, rosemary, rose, jasmine, tea tree, lemon grass, bergamot, basil, spearmint, juniper, clove, aniseed, fennel, cypress, fir, black pepper, sandalwood, cedarwood, rosewood, cardamom, cinnamon, corander, eucalyptus, geranium, ginger, chamomile, grapefruit, neroli, petitgrain, thyme, vetiver and ylang ylang.

Non-limiting examples of phase change materials include n-octacosane, n-heptacosane, n-hexacosane, n-pentacosane n-tetracosane, n-tricosane, n-docosane, n-heneicosane, n-eicosane, n-nonadecane, n-octadecane, n-heptadecane, n-hexadecane, n-pentadecane, n-tetradecane and n-tridecane.

Chemical and physical sunscreens/UV filters, e.g., 3-Benzylidene Camphor, 4-Methylbenzylidene camphor, Aminobenzoic acid (PABA)' Avobenzone, Benzophenone 4 (Sulisobenzone), Benzophenone 5, Benzophenone 8, Benzophenone-3, Benzylidene camphor sulfonic acid, Bis-ethylhexyloxyphenol methoxyphenol triazine (Escalol S), Butyl methoxy dibenzoylmethane, Camphor benzalkonium methosulfate, Cinoxate, Diethylamino hydroxybenzoyl hexyl benzoate, Dioxybenzone, Disodium phenyl dibenzimidazole tetrasulfonate, Drometrizole trisiloxane, Ensulizole, Ethylhexyl dimethyl PABA, Ethylhexyl methoxycinnamate, Ethylhexyl salicylate, Ethylhexyl triazone,

Homosalate, Isoamyl p-methoxycinnamate, Meradimate, Menthyl anthranilate, Methylene bis-benzotriazolyltetramethylbutylphenol/Bisoctrizole (Tinosorb M), Octocrylene, Octinoxate, PEG-25 PABA, Octisalate, Oxybenzone, Padimate O, Phenylbenzimidazole sulfonic acid, Polyacrylamidomethyl Benzylidene Camphor, Polysilicone-15, TEA-salicylate, Terephthalylidene dicamphor sulfonic acid, Titanium dioxide, Trolamine Salicylate and zinc oxide.

Hair treatment materials, other than those covered in the previous ingredient list. This includes cationic conditioning agents comprising tertiary and quaternary amino groups (e.g., quaternium-70, quaternium-80, stearamidopropyl dimethylamine, behentrimonium methosulfate, dicocodimonium chloride, dicetyldimonium chloride, distearyldimonium chloride hydroxyethyl cetyldimonium phosphate). Further, UV and color protectants (e.g., dimethylpabamidopropyl laurdimonium tosylate), heat protectants and styling polymers (e.g., vinyl pyrrolidone and vinylcaprolactam derivatives, such as PVP vinyl Caprolactam/DMAPA Acrylates Copolymer).

Consumer and agrichemical ingredients include insecticides and insect repellants, including, N,N-Diethyl-meta-toluamide, IR3535, Icaridin, Picaridin, Saltidin, Citronella, Permethrin, Neem oil and Lemon Eucalyptus.

Core materials also include polymeric materials, especially oil-soluble polymeric materials, which have film-forming properties on skin and hair, such as VP/Hexadecene Copolymer, Tricontanyl PVP and VP/Eicosene Copolymer as well as cosmetic and personal care actives, which are used for the conditioning or cosmetic treatment of skin, hair or nails are listed extensively and typically covered in IP.com publications IPCOM000128968D published 23 Sep. 2005 and IPCOM000133874D published 13 Feb. 2006, the contents of which are hereby incorporated by reference.

Core materials also include corrosion inhibitors which may be selected from the group consisting of carboxylic acids and derivatives such as aliphatic fatty acid derivatives, imidazolines and derivatives; including amides, quaternary ammonium salts, rosin derivatives, amines, pyridine compounds, trithione compounds, heterocyclic sulfur compounds, quinoline compounds, or salts, quats, or polymers of any of these, and mixtures thereof. For example, suitable inhibitors include primary, secondary, and tertiary monoamines; diamines; amides; polyethoxylated amines, diamines or amides; salts of such materials; and amphoteric compounds. Still other examples include imidazolines having both straight and branched alkyl chains, phosphate esters, and sulfur containing compounds.

Similarly, core materials include lipophilic scale inhibitors including those based on phosphate esters, and polyacrylates as well as oxidizing agents including inorganic or organic peroxides such as calcium peroxide, magnesium peroxides and lauryl peroxides.

As noted above, it may be desirable, if not necessary, to employ processing aids to assist in the production of the microcapsules. Two areas where processing aids are especially beneficial is in the solubilization/dispersion of the protein-based component in the water phase and in the dispersion or emulsification of the oil phase in the water phase or, if applicable, the dispersion or emulsification of the water phase in the oil phase. Generally speaking, the use of such processing aids is most beneficial where the core material is difficult to mill, particularly where it is otherwise difficult to obtain the target or desired droplet size during the emulsification process. Preferred processing aids are emulsifiers and surfactants.

Emulsifiers of all types are suitable for use in the practice of the present process though it is to be appreciated, and those skilled in the art will readily recognize that different systems, e.g., different peptides, oil phases and core materials, will be better suited with one or more classes of emulsifiers than others. Specifically, while the present teachings are applicable to anionic, cationic, non-ionic and amphoteric emulsifiers generally, preferred emulsifiers are the cationic and non-ionic emulsifiers, particularly those having polyalkylether units, especially polyethylene oxide units, with degrees of polymerization of the alkylene ether unit of greater than about 6. Preferred emulsifiers are those which significantly reduce the interfacial tension between the continuous water phase and dispersed oil phase composition, and thereby reduce the tendency for droplet coalescence. In this regard, generally the emulsifiers for use in the water phase for aiding in the oil in water emulsion or dispersion will have HLB values of from 11 to 17.

Exemplary emulsifiers include, but are not limited to polyvinyl alcohols, including PVA itself and especially those polyvinyl alcohols that are partially hydrolyzed; cellulose derivatives such as ethyl hydroxyethyl cellulose, 2-hydroxyethyl cellulose, hydroxybutyl methycellulose, hydroxypropyl methylcellulose, etc.; gums such as acacia gum and xantham gum; poly (meth) acrylic acids and derivatives; and poly (styrene-co-maleic acid) and derivatives; and the like. Most preferably, the emulsifier/emulsion stabilizer is a polyvinyl alcohol, particularly a polyvinyl alcohol that has been derived from polyvinyl acetate, wherein between 85 and 95%, preferably 88 to 90% of the vinyl acetate groups have been hydrolyzed to vinyl alcohol units.

Additional exemplary anionic surfactants and classes of anionic surfactants suitable for use in the practice of the present teaching include: sulfonates; sulfates; sulfosuccinates; sarcosinates; alcohol sulfates; alcohol ether sulfates; alkylaryl ether sulfates; alkylaryl sulfonates such as alkylbenzene sulfonates and alkylnaphthalene sulfonates and salts thereof; alkyl sulfonates; mono- or di-phosphate esters of polyalkoxylated alkyl alcohols or alkylphenols; mono- or di-sulfosuccinate esters of C₁₂ to C₁₅ alkanols or polyalkoxylated C₁₂ to C₁₅ alkanols; ether carboxylates, especially alcohol ether carboxylates; phenolic ether carboxylates; polybasic acid esters of ethoxylated polyoxyalkylene glycols consisting of oxybutylene or the residue of tetrahydrofuran; sulfoalkylamides and salts thereof such as N-methyl-N-oleoyltaurate Na salt; polyoxyalkylene alkylphenol carboxylates; polyoxyalkylene alcohol carboxylates alkyl polyglycoside/alkenyl succinic anhydride condensation products; alkyl ester sulfates; naphthalene sulfonates; naphthalene formaldehyde condensates; alkyl sulfonamides; sulfonated aliphatic polyesters; sulfate esters of styrylphenyl alkoxylates; and sulfonate esters of styrylphenyl alkoxylates and their corresponding sodium, potassium, calcium, magnesium, zinc, ammonium, alkylammonium, diethanolammonium, or triethanolammonium salts; salts of ligninsulfonic acid such as the sodium, potassium, magnesium, calcium or ammonium salt; polyarylphenol polyalkoxyether sulfates and polyarylphenol polyalkoxyether phosphates; and sulfated alkyl phenol ethoxylates and phosphated alkyl phenol ethoxylates; sodium lauryl sulfate; sodium laureth sulfate; ammonium lauryl sulfate; ammonium laureth sulfate; sodium methyl cocoyl taurate; sodium lauroyl sarcosinate; sodium cocoyl sarcosinate; potassium coco hydrolyzed collagen; TEA (triethanolamine) lauryl sulfate; TEA (Triethanolamine) laureth sulfate; lauryl or cocoyl sarcosine; disodium oleamide sulfosuccinate; disodium laureth sulfosuccinate; disodium dioctyl sulfosuccinate; N-methyl-N-oleoyltaurate Na salt; tristyrylphenol sulphate; ethoxylated lignin sulfonate; ethoxylated nonylphenol phosphate ester; calcium alkylbenzene sulfonate; ethoxylated tridecylalcohol phosphate ester; dialkyl sulfosuccinates; perfluoro (C₆-C₁₈) alkyl phosphonic acids; perfluoro (C₆-C₁₈) alkyl-phosphinic acids; perfluoro (C₃-C₂₀) alkyl esters of carboxylic acids; alkenyl succinic acid diglucamides; alkenyl succinic acid alkoxylates; sodium dialkyl sulfosuccinates; and alkenyl succinic acid alkylpolyglykosides. Further exemplification of suitable anionic emulsifiers include, but are not limited to, water-soluble salts of alkyl sulfates, alkyl ether sulfates, alkyl isothionates, alkyl carboxylates, alkyl sulfosuccinates, alkyl succinamates, alkyl sulfate salts such as sodium dodecyl sulfate (SDS), alkyl sarcosinates, alkyl derivatives of protein hydrolyzates, acyl aspartates, alkyl or alkyl ether or alkylaryl ether phosphate esters, sodium dodecyl sulphate, phospholipids or lecithin, or soaps, sodium, potassium or ammonium stearate, oleate or palmitate, alkylarylsulfonic acid salts such as sodium dodecylbenzenesulfonate, sodium dialkylsulfosuccinates, dioctyl sulfosuccinate, sodium dilaurylsulfosuccinate, poly (styrene sulfonate) sodium salt, alkylene-maleic anhydride copolymers such as isobutylene-maleic anhydride copolymer, or ethylene maleic anhydride copolymer gum arabic, sodium alginate, carboxymethylcellulose, cellulose sulfate and pectin, poly (styrene sulfonate), pectic acid, tragacanth gum, almond gum and agar; semi-synthetic polymers such as carboxymethyl cellulose, sulfated cellulose, sulfated methylcellulose, carboxymethyl starch, phosphated starch, lignin sulfonic acid; maleic anhydride copolymers (including hydrolyzates thereof), polyacrylic acid, polymethacrylic acid, acrylic acid alkyl acrylate copolymers such as acrylic acid butyl acrylate copolymer or crotonic acid homopolymers and copolymers, vinylbenzenesulfonic acid or 2-acrylamido-2-methylpropanesulfonic acid homopolymers and copolymers, and partial amide or partial ester of such polymers and copolymers, carboxymodified polyvinyl alcohol, sulfonic acid-modified polyvinyl alcohol and phosphoric acid-modified polyvinyl alcohol, phosphated or sulfated tristyrylphenol ethoxylates.

Exemplary amphoteric and cationic emulsifiers include alkylpolyglycosides; betaines; sulfobetaines; glycinates; alkanol amides of C₈ to C₁₈ fatty acids and C₈ to C₁₈ fatty amine polyalkoxylates; C₁₀ to C₁₈ alkyldimethylbenzylammonium chlorides; coconut alkyldimethylaminoacetic acids; phosphate esters of C₈ to C₁₈ fatty amine polyalkoxylates; alkylpolyglycosides (APG) obtainable from an acid-catalyzed Fischer reaction of starch or glucose syrups with fatty alcohols, in particular C₈ to C₁₈ alcohols, especially the C₈ to C₁₀ and C₁₂ to C₁₄ alkylpolyglycosides having a degree of polymerization of 1.3 to 1.6., in particular 1.4 or 1.5. Additional cationic emulsifiers include quaternary ammonium compounds with a long-chain aliphatic radical, e.g. distearyldiammonium chloride, and fatty amines. Among the cationic emulsifiers which may be mentioned are alkyldimethylbenzylammonium halides, alkyldimethylethyl ammonium halides, etc. specific cationic emulsifiers include palmitamidopropyl trimonium chloride, distearyl dimonium chloride, cetyltrimethylammonium chloride, and polyethyleneimine. Additional amphoteric emulsifiers include alkylaminoalkane carboxylic acids betaines, sulphobetaines, imidazoline derivatives, lauroamphoglycinate, sodium cocoaminopropionate, and the zwitterionic emulsifier cocoamidopropyl betaine.

Suitable non-ionic emulsifiers are characterized as having at least one non-ionic hydrophilic functional group. Preferred non-ionic hydrophilic functional groups are alcohols and amides and combinations thereof. Examples of non-ionic emulsifiers include: mono and diglycerides; polyarylphenol polyethoxy ethers; polyalkylphenol polyethoxy ethers; polyglycol ether derivatives of saturated fatty acids; polyglycol ether derivatives of unsaturated fatty acids; polyglycol ether derivatives of aliphatic alcohols; polyglycol ether derivatives of cycloaliphatic alcohols; fatty acid esters of polyoxyethylene sorbitan; alkoxylated vegetable oils; alkoxylated acetylenic diols; polyalkoxylated alkylphenols; fatty acid alkoxylates; sorbitan alkoxylates; sorbitol esters; C₈ to C₂₂ alkyl or alkenyl polyglycosides; polyalkoxy styrylaryl ethers; amine oxides especially alkylamine oxides; block copolymer ethers; polyalkoxylated fatty glyceride; polyalkylene glycol ethers; linear aliphatic or aromatic polyesters; organo silicones; polyaryl phenols; sorbitol ester alkoxylates; and mono-and diesters of ethylene glycol and mixtures thereof; ethoxylated tristyrylphenol; ethoxylated fatty alcohol; ethoxylated lauryl alcohol; ethoxylated castor oil; and ethoxylated nonylphenol; alkoxylated alcohols, amines or acids; amides of fatty acids such as stearamide, lauramide diethanolamide, and lauramide monoethanolamide; long chain fatty alcohols such as cetyl alcohol and stearyl alcohol; glycerol esters such as glyceryl laurate; polyoxyalkylene glycols and alkyl and aryl ethers of polyoxyalkylene glycols such as polyoxyethylene glycol nonylphenyl ether and polypropylene glycol stearyl ether. Polyethylene glycol oligomers and alkyl or aryl ethers or esters of oligomeric polyethylene glycol are preferred. Also preferred as non-ionic emulsifiers are polyvinyl alcohol, polyvinyl acetate, copolymers of polyvinyl alcohol and polyvinylacetate, carboxylated or partially hydrolyzed polyvinyl alcohol, methyl cellulose, various latex materials, stearates, lecithins, and various surfactants. It is known that polyvinyl alcohol is typically prepared by the partial or complete hydrolysis of polyvinyl acetate. Accordingly, by reference to polyvinyl alcohol we intend to include both completely and partially hydrolyzed polyvinyl acetate. With respect to the latter, it is preferred that the polyvinyl acetate be at least 50 mole % hydrolyzed, more preferably, at least 75 mole % hydrolyzed.

Where the emulsifier is a polymeric emulsifier, especially one having or derived from an acrylic ester, e.g., a polyacrylate, the molecular weight is generally at least 10,000, preferably at least 20,000, most preferably 30,000 or more. Additionally, the amount of emulsifier is typically from about 0.1 to about 40% by weight, more preferably from about 0.2 to about 15 percent, most preferably from about 0.5 to about 10 percent by weight based on the total weight of the formulation. It is to be appreciated that certain acrylic polymers and copolymers may perform both as an emulsifier as well as a polymerizable and/or non-polymerizable component in forming the microcapsule wall, With respect to the latter, the polymeric emulsifier, particularly those in the nature of higher molecular weight polymers, are trapped and/or incorporated into the polymer wall as it is formed. This is especially likely where the nature of the water phase changes and the solubilized polymer comes out of solution.

Though not required, it may be desirable to employ other stabilizing substances that may be used, alone or in combination with the aforementioned materials, including ionic monomers. Typical cationic monomers include dialkyl amino alkyl acrylate or methacrylate including quaternary ammonium or acid addition salts and dialkyl amino alkyl acrylamide or methacrylamide including quaternary ammonium or acid addition salts. Typical anionic monomers include ethylenically unsaturated carboxylic or sulphonic monomers such as acrylic acid, methacrylic acid, itaconic acid, allyl sulphonic acid, vinyl sulphonic acid especially alkali metal or ammonium salts. Particularly preferred anionic monomers are ethylenically unsaturated sulphonic acids and salts thereof, especially 2-acrylamido-2-methyl propane sulphonic acid, and salts thereof.

Similarly, though not necessary, the water phase compositions and the core phase compositions may further contain other ingredients conventional in the art including, e.g., chain transfer agents and/or agents which help control the molecular weight/degree of polymerization of the wall forming monomer, thereby aiding in the movement of the oligomer/prepolymer through the respective oil phase and water phase compositions. Suitable chain transfer agents include, but are not limited to, lower alkyl alcohols having from 1 to 5 carbon atoms, mercaptoethanol, mercaptopropanol, thioglycolic acid, isooctylmercaptoproprionate, tert-nonylmercaptan, pentaerythritol tetrakis (3-mercaptoproprionate). dodecylmercaptan. formic acid, halogenated hydrocarbons, such as bromoethane, bromotrichloromethane, or carbon tetrachloride, and the sulfate, bisulfate, hydrosulfate, phosphate, monohydrogen phosphate, dihydrogen phosphate, toluene sulfonate, and benzoate salts of sodium and potassium, especially sodium hypophosphite and sodium bisulfate. If present, the chain transfer agents are preferably used in amounts ranging from 0.01 to 5%, preferably from 0.5 to 3%, by weight with respect to the wall forming monomers and/or oligomers employed.

Following on the foregoing, the wall forming composition may also include various polyfunctional amines and alcohols which can be dispersed or dissolved in water or an aqueous solution and are capable of reacting with the isocyanate and/or the chloro-based crosslinking agent, e.g., chloroformate, especially the isocyanate, to serve as supplemental cross-linkers for modifying the microcapsule wall physical properties. In particular, the preferred supplemental cross-linkers can be employed to manipulate or control release characteristics while having minimal or modest impact upon degradability. Such cross-linking agents are well known in the art and generally have two or more, preferably two to five, primary or secondary amine groups or hydroxy groups or a combination of hydroxy and amine groups, Generally, they may be individual compounds, dimers, oligomers or low molecular weight polymers. Most especially, they tend to be lower molecular weight, generally having molecular weights of 500 or less, preferably 250 or less. Exemplary amines include 1,2-ethylenediamine, 1,3-diamino propane, 1,4-diaminobutane, 1,6-diaminohexane, hydrazine, 1,4-diamino-cyclohexane, 1,3-diamino-1-methylpropane, diethylenetriamine, triethylenetetramine, tetraethylene-pentamine, bis(2-methylamino-ethyl)methylamine, triethanolamine, bis(dimethylamino-ethyl)ether, triisopropanolamine, ethanolamine, guanidine amine and its derivatives, etc. Exemplary polyols include ethylene glycol, propylene glycol, diethylene glycol, dipropylene glycol, triethylene glycol, 2-ethylhexanediol-1,3,glycerin, 1,2,6-hexane triol, trimethylol propane, trimethylol ethane, and tris(hydroxy-phenyl)propane. If present, these supplemental cross-linking agents are preferably used in amounts ranging from 0.01 to 20%, preferably from 0.5 to 10%, by weight with respect to the wall forming monomers and/or oligomers employed. These supplemental cross-linkers may be added to the water phase prior to formation of the emulsion or to the emulsion once formed.

The particle size of the microcapsules of the present teaching will vary widely depending upon the core material as well as the intended end-use application, and the constraints of the method by which the microcapsules, especially the core material dispersion/emulsion, is formed. Typically, the volume weighted median particle size will range from about 2 microns to about 200 microns, preferably from about 5 microns to about 50 microns, most preferably from about 10 microns to about 20 microns. Suitable equipment for making such measurements are widely available, including, e.g., Accusizer 780A, made by Particle Sizing Systems, Santa Barbara, CA.

In following, the thickness of the microcapsule walls is likewise dependent upon the intended end-use of the microcapsules and is influenced, as well, by the wall forming materials and the level of their concentration and the physical properties of/desired of the microcapsule walls. Wall thickness is generally controlled by the amount of wall forming materials employed in the encapsulation process as well as the size/quantity of the beads of the dispersed phase to be encapsulated. Generally speaking, the wall forming materials comprise from 3 to 30, preferably from 5 to 20, more preferably from 8 to 15, weight percent of the microcapsule forming materials, i.e., wall forming materials and core materials.

Microencapsulation of the core material with the wall forming materials as described above may be attained through any of the known methods for microencapsulation. Suitable techniques include coacervation, interfacial polymerization, air suspension, centrifugal extrusion, spray drying, pan coating, in-situ polymerization, and by forming a dispersion of core material and shell material and applying a pressure shock wave to the dispersion as described in Redding Jr. (U.S. Pat. No. 5,271,881, incorporated herein by reference). The specific selection of the method and the materials depends upon the nature, including the physical state and/or chemistry, of the material to be encapsulated, e.g., whether the carrier material is in a liquid form or a solid, semi-solid or gel-like particulate form. Exemplary methods are set forth in the following paragraphs as well as in, for example, Schwantes (U.S. Pat. No. 6,592,990), Nagai et. al. (U.S. Pat. No. 4,708,924), Baker et. al. (U.S. Pat. No. 4,166,152), Wojciak (U.S. Pat. No. 4,093,556), Matsukawa et. al. (U.S. Pat. No. 3,965,033), Matsukawa (U.S. Pat. No. 3,660,304), Ozono (U.S. Pat. No. 4,588,639), Irgarashi et. al. (U.S. Pat. No. 4,610,927), Brown et. al. (U.S. Pat. No. 4,552,811), Scher (U.S. Pat. No. 4,285,720), Shioi et. al. (U.S. Pat. No. 4,601,863), Kiritani et. al. (U.S. Pat. No. 3,886,085), Jahns et. al. (U.S. Pat. No. 5,596,051 and 5,292,835), Matson (U.S. Pat. No. 3,516,941), Chao (U.S. Pat. No. 6,375,872), Foris et. al. (U.S. Pat. Nos. 4,001,140; 4,087,376; 4,089,802 and 4,100,103), Greene et. al. (U.S. Pat. No. 2,800,458 and 2,730,456), Clark (U.S. Pat. No. 6,531,156), Saeki et. al. (U.S. Pat. No. 4,251,386 and 4,356,109), Hoshi et. al. (U.S. Pat. No. 4,221,710), Hayford (U.S. Pat. No. 4,444,699), Hasler et. al. (U.S. Pat. No. 5,105,823), Stevens (U.S. Pat. No. 4,197,346), Riecke (U.S. Pat. No. 4,622,267), Greiner et. al. (U.S. Pat. No. 4,547,429), and Tice et. al. (U.S. Pat. No. 5,407,609), among others and as taught by Herbig in the chapter entitled “Encapsulation” in Kirk Othmer, Encyclopedia of Chemical Technology, V.13, Second Edition, pages 436-456 and by Huber et. al. in “Capsular Adhesives”, TAPPI, Vol. 49, No. 5, pages 41A-44A, May 1966, all of which are incorporated herein by reference.

Generally speaking, the first step in the encapsulation process is the preparation of discrete particles, domains, droplets or beads of the core material, with or without one of the wall forming components and/or processing aids (e.g., emulsifiers), depending upon the specific core material and the process to be used, eg., water-in-oil, oil-in-water, water-in-oil-in-water. Typically, the core material is dissolved in, solubilized in, dispersed in, suspended in, emulsified in one of the wall forming materials, which, alone and/or with another solvent or carrier or processing aid, is employed as one of the phases of the microencapsulation process. Where such materials are in solution or liquid form and the encapsulation is to be by way of, e.g., coacervation, interfacial polymerization, etc., the solution or liquid containing the core material is subjected to high shear mixing or agitation to create a suspension, emulsion or colloidal system of discrete domains of the core material of the requisite size. Where the core material is a heat sensitive material, e.g., a wax or wax-like material, the carrier, with the therein incorporated core material, is heated above its melt temperature and then subjected to a similar high shear mixing or agitation in a liquid medium, preferably one of the wall-forming materials or the phase material, e.g., water or oil phase, to create discrete droplets of the core material and then cooled to allow the solid particles to form, before encapsulating. Where the core material is a solid or substantially solid material, it may be ground and sorted to the desired particle size before encapsulation. Such methods, as well as additional alternative methods for preparation of the particles or discrete domains for encapsulation are widely used in industry and well known to those skilled in the art.

Although not limited thereto, the microcapsules according to the present teaching are preferably prepared by interfacial polymerization. Here, the core material is solubilized, dispersed or emulsified in a liquid solution of one of the materials to be used as/containing one of the wall forming materials, preferably the oil phase comprising the isocyanate, or, in the case of a water-in-oil-in-water system, in a first water phase, preferably free of wall forming material, which water phase containing the core material is then dispersed or emulsified in the oil phase which, in turn, is dispersed or emulsified in a second water phase, which is ultimately the continuous phase for the interfacial polymerization and contains the other wall forming material, namely the peptide and/or proteins.

As noted above, the microcapsules made in accordance with the present teaching have a myriad of commercial and consumer applications across most all industries and in many commercial products.

In one embodiment, microcapsules described herein may be incorporated in personal care products and compositions including, but not limited to, cosmetics, drug delivery systems, hair care products, skin treatment products and oils, pharmaceuticals, pigment dispersions, preservative compositions, skin coloring products, skin restorative products, styling products for hair, sunscreen and suntan lotions, sprays, oils, creams and the like, water proof/resistance products, wear resistance products and additives, shower gels, shampoos, and thermal protecting/enhancing compositions. Dental personal care compositions include denture adhesives, toothpastes, mouth washes, chewing gums and the like.

The microcapsules may be used in numerous pharmaceutical applications and compositions including peroral and topical dosage forms, such as tablets, pellets, capsules, dermatological products (creams, gels, ointments, sprays, lotions, and foams), transdermal patches and the like.

The microcapsules may also be used in conjunction with a myriad of agrochemicals, especially those listed extensively in U.S. Pat. No. 5,389,688, to ISP, which is incorporated herein by reference in its entirety.

In common consumer products, the microcapsules may be used to incorporate actives such as fabric conditioners, liquid laundering detergents, powdered laundering detergents, dish washing detergents, hard surface cleaners, anti-static agents, anti-odor agents, antimicrobial agents, etc., into various household cleaners and other “cleaning” products such as air fresheners, sprays, and the like, as well as onto textiles, paper and the like as surface modifiers or coatings.

The microcapsules of the present teaching can be advantageously used in controlling perfume release in fragrant consumer products. With the microcapsules of the present teaching there is a considerable improvement in longevity and intensity of the encapsulated perfume in actual use. Examples of consumer products comprising perfume microcapsules according to certain aspects of the present application may fall into product group categories of laundering detergents, cosmetics, personal care products, dish washing detergents and house cleaners. More specific examples of consumer products include fabric conditioners, liquid/powdered laundering detergents, dish washing detergents, hair shampoos, hair conditioners, hair styling gels, soaps, body washes, shower gels, all-purpose cleaners including hard surface cleaners, carpet cleaners, body lotions, antiperspirant/deodorants and spray-able products.

In following, in another embodiment of the present teaching there is provided a method for producing fragrance loaded microcapsules with improved substantivity for incorporation into, (i) laundry detergents; (ii) fabric softener compositions; and (iii) drier-added fabric softener articles, these when deposited on fabrics during laundry treatment and capable of remaining on the textile following initial application and which is capable of later being sheared by the application of mechanical force. Accordingly, the encapsulated fragrance provides a “burst” of fragrance during wear and/or cleaning due to breakage of the capsule wall.

Alternatively, the fragrance microcapsules of the present application can be formulated into solid fabric care compositions with polysaccharides such as sugars according to the procedure described in US Patent No 2011/0082066, the contents of which are hereby incorporated by reference. The solid fabric care products can be used for delivering fragrances onto the textile articles during the washing/cleaning cycle and subsequently the laundered textiles have beneficial fragrance odor profile during the wear.

Alternatively, the fragrance microcapsules can be incorporated in 2-in-1 powdered detergent and conditioner compositions according to the processes described in U.S. Pat. Nos. 4,698,167 and 5,540,850 and also crystalline laundry additives as described in the US application 2011/97369 and PCT WO 2010/000558, which are incorporated herein by reference.

For some embodiments, it may be preferred to incorporate a plurality of core materials into a single microcapsule and/or provide mixtures of such microcapsules, For example, one may add one or more preservatives and/or antimicrobial agents in the delivery matrix in addition to the respective actives, such as, but not limited to, benzoic acid, sorbic acid, dehydroacetic acid, piroctone olamine, DMDM hydantoin, IPBC, triclosan, bronopol, isothiazolinones, parabens, phenoxyethanol, and combination thereof.

In another embodiment, the core material may be a phase change material or mixtures thereof for temperature control. Typical phase change materials exhibit a melting temperature from −20° C. to 100° C. and generally comprise linear or branched hydrocarbons or fatty esters or mixtures thereof, of different chain lengths and melting points. The microcapsules containing the phase change material core material can be coated or sprayed onto or incorporated into suitable materials including textile fibers, such as cotton and polyester, during the spinning process or coated directly onto textiles or incorporated into building construction material for example bricks, gypsum, and the like, to allow for temperature control by use of latent heat of fusion.

It is further contemplated that microcapsules of the present teaching can be used in the construction industry in conjunction with cements, plaster boards, breeze blocks, chipboards, heat transfer fluids, sealants, adhesives etc. In this instance, the core material can be a phase change material, biocide, flame retardant, catalyst, epoxy resin, etc.

The present microcapsules also have a number of automotive applications including the use of encapsulated phase change materials in the coolant systems, the use of encapsulated lubricant additives such as anti-wear additives in engine oils, and the use of encapsulated UV absorbers and/or anti-corrosive agents for car coatings.

The microcapsules described herein may also be used in conjunction with additives used in plastics such as flame retardants, catalysts, pigments, light stabilizers, UV absorbers, and the like, all of which can be encapsulated to allow higher compatibilities, longevity and self-healing of the plastic material. For example, the core material can be a catalyst for self-healing, an UV absorber for protection from photodamage due to UV light, or a thermochromic material for color change in coatings across a broad spectrum of industry.

The microcapsules according to the present teaching may also be used in oilfield applications. Here the microcapsules may contain traditional oilfield chemicals such as corrosion inhibitors, scale inhibitors, oxidizing agents, crosslinking agents, catalysts, acidizing agents, biocides, demulsifiers, enzymes, polymers, lubricants, shale inhibitors, solvents, and surfactants. The encapsulated oil field chemicals can be applied advantageously at the different petroleum extraction stages from drilling, cementing, stimulation to production and enhanced oil recovery. The release mechanisms of delivery of the oilfield chemical can be by temperature, dilution, pH and shear at the relevant points of applications.

### EXAMPLES

A number of microcapsules were formed in accordance with the present teaching as well as a number of comparative microcapsules using materials outside of the scope of the limitations set forth herein. Typically, the microcapsules were formed in accordance with the following steps using non-water soluble core materials:
An aqueous phase solution was prepared by dissolving with agitation the protein-based component, with or without an emulsifier, in deionized water in a stainless steel reactor vessel equipped with a mixing blade at ambient temperature. Once the mixture was prepared, the mixing blade was replaced with a milling blade.
An oil phase solution was prepared by mixing the core material to be encapsulated with the cross-linking component(s) with a mixer blade in another vessel at ambient temperature.
The oil phase solution was then gradually added to the water phase solution under high shear milling at a predetermined temperature to form an oil-in-water emulsion with the high shear milling continuing until the targeted emulsion droplet size was attained.
Once the targeted droplet size was attained, milling was paused and the milling blade replaced with a mixer blade and the blade activated to continuously agitate the mixture to maintain the emulsion.
Thereafter the temperature of the reaction vessel was raised to a temperature of 50° C. over a period of 60 minutes and maintained at that temperature for an additional 60 minutes.
Subsequently, the temperature of the reaction vessel was raised to a temperature of 65° C. over a period of 60 minutes and maintained at that temperature for a period of 120 minutes.

Thereafter, the temperature of the reaction vessel was raised to a temperature of 85° C. over a period of 60 minutes and maintained at that temperature for an additional 6 hours, at which point the reaction was deemed completed.
At the end of the reaction, the resulting microcapsule slurry was allowed to cool to ambient temperature in the reactor vessel. The slurry was found to be of low viscosity and generally uniform and smooth with no visible thickening and/or agglomeration. Microcapsules were subsequently recovered from the slurry for testing.

Although the preparation and milling of the solutions and mixture is preferably done at room temperature, it is to be appreciated that somewhat elevated temperatures may be desirable to aid in quicker and easier formation of the solutions and/or the emulsion. Furthermore, one may vary the milling temperature and/or the rate of temperature ramp up during wall formation depending on the reactivity and quantity of the raw materials used. In this respect, it is to be appreciated that the capsule slurry may become gelled, or emulsion become coalesced during temperature ramp up and/or wall formation if too high of a milling temperature and/or to fast a rate of temperature ramp up are used. In this respect, milling at room temperature is preferred if the raw materials are very reactive. On the other hand, higher milling temperature is preferred if the raw materials are less reactive.

The resulting microcapsules were evaluated for release of a core fragrance oil from the microcapsules in a hexane release/extraction as well as in two traditional end-use products for such microcapsules, namely a heavy duty laundry detergent (HDL) and a liquid fabric enhancer (LFE): the former from Seventh Generation, Inc. and the latter unscented Downy® fabric softener. All tests were performed in duplicate or triplicate.

### Release of Fragrance Oil in Hexane

The premise of this method is based on the CIPAC MT190. The capsule slurry is placed into a 110 ml jar. To this, hexane with an internal standard (dicyclohexyl phthalate) is added to the jar. The jar is placed on a horizontal roller, rolling at 70 RPM. At time intervals of 15, 30, 60, and 180 minutes, exactly 1 ml of solution is removed and placed into an injection vial. The injection vial is then analyzed via GC-FID for the core of interest.

### Release of Fragrance Oil RA Into Liquid Matrices

The microcapsules were evaluated for release properties, i.e., release of the fragrance, in a heavy duty liquid laundry detergent (HDL) from Seventh Generation, Inc. and in a commercial liquid fabric enhancer (LFE-unscented Downy® fabric softener). A sample of LFE or HDL is weighed into a 50ml glass jar. To this, a specified amount of capsule slurry is added and thoroughly mixed. After mixing, the jar is tightly capped and placed into an oven at 35° C. for one week. After the week has elapsed, the sample is removed from the oven. A sample aliquot is removed from the jar into a Scintillation vial. A small amount of water is added and gently mixed, ensuring no foam is generated. To this, hexane is added to extract the core for analysis via minor mixing. The solution is allowed to sit to separate and then the hexane layer is sampled to be analyzed via GC-FID.

### Example 1 (Comparative Example)

A microcapsule was formed in accordance with the aforementioned process. Specifically, a water phase solution was prepared by dissolving 13.6 grams of soy protein isolate from MP Biomaterials, LLC. in 217 grams of deionized water in a 1-liter stainless reactor. A capsule core composition was prepared in a beaker at ambient temperature by mixing 81.4 grams of a fragrance blend Research Accord (RA) comprising a combination of common odiferous compounds including benzyl acetate, isobornyl acetate, hexyl salicylate, and the like, with 81.4 grams of Captex® 355 caprylic/capric triglyceride from ABITEC Corporation (fragrance diluent). Subsequently, 6.78 grams of Mondur® MR Light isocyanate (polymeric MDI (methylene diphenyl diisocyanate) from Covestro LLC was then added to the beaker under agitation until thoroughly mixed to form the oil phase composition. The oil phase composition was then gradually added to the reactor containing the water phase composition under high shear milling and the high shear milling maintained until the targeted emulsion droplet size of around 10 microns was attained. Once the target emulsion droplet size was achieved, milling was paused, and the milling blade switched out for the mixer blade to keep the emulsion mixed and the mixture subjected to the elevated temperature reaction steps set forth above to form the microcapsules.

### Example 2

A microcapsule slurry was prepared in accordance with the process of Example 1 except that 6.44 grams of Mondur® MR Light isocyanate and 0.34 grams of sebacoyl chloride from Sigma Aldrich were employed as the crosslinking component.

### Example 3

A microcapsule slurry was prepared in accordance with the process of Example 1 except that 6.1 grams of Mondur MR Light isocyanate and 0.68 grams of sebaocyl chloride from Sigma Aldrich were employed as the crosslinking component.

### Example 4

A microcapsule slurry was prepared in accordance with the process of Example 1 except that 5.42 grams of Mondur® MR Light isocyanate and 1.36 grams of sebacoyl chloride from Sigma Aldrich were employed as the crosslinking component.

### Example 5

A microcapsule slurry was prepared in accordance with the process of Example 1 except that 6.44 grams of Mondur® MR Light isocyanate and 0.34 grams of adipoyl chloride from TCI America were employed as the crosslinking component.

### Example 6

A microcapsule slurry was prepared in accordance with the process of Example 1 except that 6.1 grams of Mondur® MR Light isocyanate and 0.68 grams of adipoyl chloride from TCI America were employed as the crosslinking component.

### Example 7

A microcapsule slurry was prepared in accordance with the process of Example 1 except that 5.42 grams of Mondur® MR Light isocyanate and 1.36 grams of adipoyl chloride from TCI America were employed as the crosslinking component.

### Example 8

A microcapsule slurry was prepared in accordance with the process of Example 1 except that 6.44 grams of Mondure MR Light isocyanate and 0.34 grams of tri(ethylene glycol) bis(chloroformate) from Sigma Aldrich were employed as the crosslinking component.

### Example 9

A microcapsule slurry was prepared in accordance with the process of Example 1 except that 6.1 grams of Mondur® MR Light isocyanate and 0.68 grams of tri(ethylene glycol) bis(chloroformate) from Sigma Aldrich were employed as the crosslinking component.

### Example 10

A microcapsule slurry was prepared in accordance with the process of Example 1 except that 5.42 grams of Mondur® MR Light isocyanate and 1.36 grams of tri(ethylene glycol) bis(chloroformate) from Sigma Aldrich were employed as the crosslinking component.

### Example 11

A microcapsule slurry was prepared in accordance with the process of Example 1 except that 6.44 grams of Mondur® MR Light isocyanate and 0.34 grams of bisphenol A bis(chloroformate) from Sigma Aldrich were employed as the crosslinking component.

### Example 12

A microcapsule slurry was prepared in accordance with the process of Example 1 except that 6.1 grams of Mondur® MR Light isocyanate and 0.68 grams of bisphenol A bis(chloroformate) from Sigma Aldrich were employed as the crosslinking component.

### Example 13

A microcapsule slurry was prepared in accordance with the process of Example 1 except that 5.42 grams of Mondur® MR Light isocyanate and 1.36 grams of bisphenol A bis(chloroformate) from Sigma Aldrich were employed as the crosslinking component.

### Example 14

A microcapsule slurry was prepared in accordance with the process of Example 1 except that 6.44 grams of Mondur® MR Light isocyanate and 0.34 grams of 1,3 benzenedisulfonyl chloride from TCI America were employed as the crosslinking component.

### Example 15

A microcapsule slurry was prepared in accordance with the process of Example 1 except that 6.1 grams of Mondur® MR Light isocyanate and 0.68 grams of 1,3 benzenedisulfonyl chloride from TCI America were employed as the crosslinking component.

### Example 16

A microcapsule slurry was prepared in accordance with the process of Example 1 except that 5.42 grams of Mondur MR Light isocyanate and 1.36 grams of 1,3 benzenedisulfonyl chloride from TCI America were employed as the crosslinking component.

### Example 17

A microcapsule slurry was prepared in accordance with the process of Example 1 except that 6.78 grams of Takenate D-110N isocyanate (xylene diisocyanate adduct polymer) from Mitsui Chemicals, Inc. was employed as the crosslinkingomponent.

### Example 18

A microcapsule slurry was prepared in accordance with the process of Example 17 except that 6.44 grams of Takenate D-110N isocyanate and 0.34 grams of sebacoyl chloride from Sigma Aldrich were employed as the crosslinking component.

### Example 19

A microcapsule slurry was prepared in accordance with the process of Example 17 except that 6.1 grams of Takenate D-110N isocyanate and 0.68 grams of sebacoyl chloride from Sigma Aldrich were employed as the crosslinking component.

### Example 20

A microcapsule slurry was prepared in accordance with the process of Example 17 except that 5.42 grams of Takenate D-110N isocyanate and 1.36 grams of sebacoyl chloride from Sigma Aldrich were employed as the crosslinking component.

Table 1 presents an overview of the formulation of each set of the foregoing microcapsules prepared with two crosslinkers and the fragrance release from the capsules in hexane as well as in HDL and LFE matrices.

### Example 21 (Comparative Example)

A microcapsule was formed in accordance with the process as follows. A water phase solution was prepared by dissolving 13.6 grams of gelatin from Ajinomoto North America, Inc. in 217 grams of deionized water in a 1-liter stainless reactor. A capsule core composition was prepared in a beaker at ambient temperature by mixing 138 grams of a fragrance blend Research Accord (RA) comprising a combination of common odiferous compounds including benzyl acetate, isobornyl acetate, hexyl salicylate, and the like, with 24 grams of Captex® 355 caprylic/capric triglyceride from ABITEC Corporation (fragrance diluent). Subsequently, 6.78 grams of Takenate D110N isocyanate (xylene diisocyanate adduct polymer) from Mitsui Chemicals, Inc was then added to the beaker under agitation until thoroughly mixed to form the oil phase composition. The oil phase composition was then gradually added to the reactor

TABLE 1

| | | | Leakage at 35° C. in one week | | | | | | |
|---|---|---|---|---|---|---|---|---|---|
| | | % Co- | 7th Gen | Downy | % Release of RA in hexane, min | | | | |
| ID | Crosslinkers | crosslinker | HDL | LFE | 0 | 15 | 30 | 60 | 180 |
| 1 | Mon#Z.899;ur MR Light (MMR) | 0.0 | 63.60 | 29.44 | 0 | 27.10 | 30.37 | 34.27 | 46.85 |
| 2 | MMR, 5%Sebacoyl chloride | 5.0 | 54.95 | 27.3 [symbol] | 0 | 24.95 | 27.06 | 32.24 | 39.47 |
| 3 | MMR, 10%Sebacoyl chloride | 10.0 | 50.91 | 25.60 | 0 | [symbol] .79 | 18.75 | 22.97 | 30.91 |
| 4 | MMR, 20%Sebacoyl chloride | 20.0 | 65.27 | [symbol] 5.4 [symbol] | 0 | 20.12 | 23.87 | 28.54 | [symbol] 8.58 |
| 1 | MMR | 0.0 | [symbol] 3.60 | 29.44 | 0 | 27.10 | 30.3 [symbol] | 34. [symbol] 7 | 46.85 |
| 5 | MMR, 5%Adipoyl chloride | 5.0 | 43. [symbol] | 25.73 | 0 | 16.38 | 19.58 | 2 [symbol] .56 | [symbol] 2.37 |
| 6 | MMR, 10%Adipoyl chloride | 10.0 | [symbol] 0. [symbol] | 43.97 | 0 | 32.6 [symbol] | [symbol] 7.74 | 43. [symbol] 7 | 61.83 |
| 7 | MMR, 20%Adipoyl chloride | 20.0 | 83. [symbol] 4 | 44.84 | 0 | 24.21 | 28.55 | 33. [symbol] 3 | 44.64 |
| 1 | MMR | 0.0 | 63.60 | 29.44 | 0 | 27.10 | 30.37 | 34.27 | 46.8 [symbol] |
| 8 | MMR, 5% Tr [symbol] (EG) bis(Chloroformate) | 5.0 | 45.46 | [symbol] 4.20 | 0 | 18. [symbol] 1 | 20.92 | 25.63 | 3 [symbol] .93 |
| 9 | MMR, 10% Tr [symbol] (EG) bis(Chloroformate) | 10.0 | [symbol] .92 | 3 [symbol] .14 | 0 | 45. [symbol] 4 | 51.87 | 58. [symbol] 9 | 6 [symbol] .24 |
| 10 | MMR, 20% Tr [symbol] (EG) bis(Chloroformate) | 20.0 | 98.61 | 44.31 | 0 | [symbol] 9.92 | 68.25 | 73.18 | 86. [symbol] 7 |
| 1 | MMR | 0.0 | [symbol] 3.60 | 29.44 | 0 | 27.10 | 30.37 | 34.27 | 46.85 |
| 11 | MMR, 5%Biphenol A bis(Chloroformate) | 5.0 | 46.83 | 24.80 | 0 | 19.01 | 22.09 | 27.76 | 38.52 |
| 12 | MMR, 10%Biphenol A bis(Chloroformate) | 10.0 | [symbol] 0.58 | 24.5 [symbol] | 0 | 13. [symbol] | 16.87 | 21.26 | 34. [symbol] 5 |
| 13 | MMR, 20%Biphenol A bis(Chloroformate) | 20.0 | [symbol] 5.91 | 48.93 | 0 | [symbol] 4.3 [symbol] | [symbol] .44 | 62.58 | 72.64 |
| 1 | MMR | 0.0 | 63.60 | 29.44 | 0 | 27.10 | 30.3 [symbol] | 34.27 | 46.85 |
| 14 | MMR, 5%/1,3Benzenedisulfonyl Chloride | 5.0 | [symbol] 8.80 | 29.74 | 0 | 13. [symbol] 5 | 16.88 | 2 [symbol] . [symbol] | 29.88 |
| 15 | MMR, 10%/1,3Benzenedisulfonyl Chloride | 10.0 | 60.28 | 30.92 | 0 | 17.22 | 1 [symbol] .31 | 23. [symbol] 7 | 31.31 |
| 16 | MMR, 20%/1,3Benzenedisulfonyl Chloride | 20.0 | 73.42 | [symbol] 2.00 | 0 | 31.69 | 37.74 | 43.39 | 54.12 |
| 17 | Takenate D-110N (TD) | 0.0 | 62.38 | 3 [symbol] .84 | 0 | 20.12 | 22.12 | 24.8 [symbol] | 30.67 |
| 18 | TD, 5%Sebacoyl chloride | 5.0 | 4 [symbol] .85 | 23.36 | 0 | 25.31 | 28.24 | 31. [symbol] 1 | 36.54 |
| 19 | TD, 10%Sebacoyl chloride | 10.0 | [symbol] 4.47 | 35.91 | 0 | 26.00 | 30.63 | 36.11 | 44.20 |
| 20 | TD, 20%Sebacoyl chloride | 20.0 | 87.41 | 48.36 | 0 | [symbol] .25 | 62.14 | 68.11 | 7 [symbol] .9 [symbol] |

[symbol]

indicates data missing or illegible when filed

containing the water phase composition under high shear milling and the high shear milling maintained until the targeted emulsion droplet size of around 10 microns was attained. Once the target emulsion droplet size was achieved, milling was paused, and the milling blade switched out for the mixer blade to keep the emulsion mixed and the mixture subjected to the elevated temperature reaction steps set forth above to form the microcapsules.

### Example 22

A microcapsule slurry was prepared in accordance with the process of Example 21 except that 6.44 grams of Takenate D-110N isocyanate and 0.34 grams of sebacoyl chloride from Sigma Aldrich were employed as the crosslinking component.

### Example 23

A microcapsule slurry was prepared in accordance with the process of Example 21 except that 6.1 grams of Takenate D-110N isocyanate and 0.68 grams of sebacoyl chloride from Sigma Aldrich were employed as the crosslinking component.

### Example 24

A microcapsule slurry was prepared in accordance with the process of Example 21 except that 5.42 grams of Takenate D-110N isocyanate and 1.36 grams of sebacoyl chloride from Sigma Aldrich were employed as the crosslinking component.

Table 2 presents an overview of the formulation of each set of the foregoing microcapsules of Examples 21 through 24 prepared with Takenate D-110N isocyanate and sebacoyl chloride as crosslinkers and the fragrance release from the capsules in hexane.

TABLE 2

| | | % Release of RA in hexane, min | | | | |
|---|---|---|---|---|---|---|
| ID | Crosslinker | 0 | 15 | 30 | 60 | 180 |
| 21 | Takenate D110N (TD) | 0.00 | 2.11 | 2.26 | 2.45 | 2.99 |
| 22 | TD, 5%Sebacoyl chloride | 0.00 | 1.6 | 1.66 | 1.79 | 2.15 |
| 23 | TD, 10%Sebacoyl chloride | 0.00 | 1.85 | 1.9 | 2.05 | 2.35 |
| 24 | TD, 20%Sebacoyl chloride | 0.00 | 4.12 | 4.61 | 5.67 | 6.48 |

The results shown in Tables1 and 2 demonstrate the ability to establish and control release properties of the microcapsules, essentially create custom release microcapsules, based upon the selection of wall forming materials and their relative concentrations. In this respect, once a small sample/library establishing the correlation between the select wall forming components and their relative concentrations and the release properties of the given microcapsules, one can then customize the microcapsules by specific selection of the wall forming component and their amounts for a given release profile.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. No unclaimed language should be deemed to limit the invention in scope. Any statements or suggestions herein that certain features constitute a component of the claimed invention are not intended to be limiting unless reflected in the appended claims.

Although the process and prepared microcapsules of the present specification have been described with respect to specific embodiments and examples, it should be appreciated that the present teachings are not limited thereto and other embodiments utilizing the concepts expressed herein are intended and contemplated without departing from the scope of the present teaching. Thus, the true scope of the present teachings is defined by the claimed process steps and any and all modifications, variations, or equivalents that fall within the spirit and scope of the underlying principles set forth herein.

I claim:

1. A microcapsule comprising the reaction product of
A) one or more protein-based components selected from proteins, peptides, polypeptides and combinations of any two or more of the foregoing, and
B) a cross-linking component comprising a combination of two or more cross-linking agents, at least one of which is i) an isocyanate component selected from the group consisting of one or more diisocyanates and/or poly-isocyanates and at least one of which is ii) a chloro component selected from the group consisting of a) di- and/or poly-acid chlorides, b) bis- and/or poly-chloroformates and/or c) di- and/or poly-sulfonyl chlorides;
wherein (I) the weight ratio of the protein-based components (A) to the cross-linking component (B) is from 100:1 to 1:100 ((A): (B)) and (II) the weight ratio of the isocyanate component (i) to the chloro component (ii) is from 100:1 to 100:50 (i):(ii)).

2. The microcapsule of claim 1 wherein the weight ratio of the protein-based components (A) to the cross-linking component (B) is from 10:1 to 1:10.

3. The microcapsule of claim 1 wherein the weight ratio of the protein-based components (A) to the cross-linking component (B) is from 1:5 to 1:0.2.

4. The microcapsule of claim 1 wherein the weight ratio of the isocyanate component (i) to the chloro component (ii) is from 100:5 to 100:15 ((i):(ii))

5. The microcapsule of claim 1 wherein the protein-based component is an animal-based protein component or a synthetic equivalent thereof.

6. The microcapsule of claim 5 wherein the animal-based protein component is selected from proteins, polypeptides, peptides, egg protein, fibroin, a dairy protein, gelatin and combinations of one more of the foregoing.

7. The microcapsule of claim 1 wherein the protein-based component is a plant-based protein component or a synthetic equivalent thereof.

8. The microcapsule of claim 1 wherein the protein component is a protein of from 10,000 Da to 110,000 Da.

9. The microcapsule of claim 1 wherein the protein component is a protein of from 15,000 Da to about 40,000 Da.

10. The microcapsule of claim 1 wherein the protein component is a polypeptide of at least two amino acids and a weight of less than 10,000 Da.

11. The microcapsule of claim 10 wherein the polypeptides comprise from 5 to 50 amino acids.

12. The microcapsule of claim 1 wherein the protein component is a starting protein which has been subjected to fragmentation whereby the median particle size D50 of the starting protein has been reduced by at least about 40%.

13. The microcapsule of claim 1 wherein the protein component is a starting protein which has been subjected to fragmentation whereby the median particle size of the fragmented protein is from about 0.1% to about 60% of the median particle size of the starting protein prior to fragmentation.

14. The microcapsule of claim 1 wherein the isocyanate component is an aliphatic. cycloaliphatic, aromatic, or polyaromatic isocyanate or a dimer, trimer, oligomer or prepolymer thereof or combination of two or more such isocyanates.

15. The microcapsule of claim 1 wherein the isocyanate has from 2 to 16 carbon atoms in the basic hydrocarbon skeleton and from 2 to 4 cyanato (NCO) groups.

16. The microcapsule of claim 15 wherein the isocyanate component is a di-isocyanate.

17. The microcapsule of claim 15 wherein the isocyanate component is a combination of di- and/or poly-isocyanates.

18. The microcapsule of claim 1 wherein the isocyanate component is a combination of aliphatic and aromatic isocyanates.

19. The microcapsule of claim 1 wherein the isocyanate component is a combination of two or more isocyanates wherein each is present in an amount of at least 10% by weight.

20. The microcapsule of claim 1 wherein the chloro component is one or more acid chlorides.

21. The microcapsule of claim 20 wherein the acid chlorides have from 4 to 12 carbon atoms and are selected from aliphatic, saturated or unsaturated, straight chain or branched chain polyacids.

22. The microcapsule of claim 20 wherein the acid chloride is selected from adipoyl chloride, octanedioyl dichloride, azelaoyl chloride, sebacoyl chloride, dodecanedioyl dichloride, isophthaloyl chloride, terephthaloyl chloride, 1,3,5-benzenetricarbonyl chloride, 1,2,4,5-benzenetetracarbonyl tetrachloride, and combinations or any two or more of the foregoing.

23. The microcapsule of claim 1 wherein the chloro component is one or more bis- and/or poly-chloroformates.

24. The microcapsule of claim 23 wherein the chloroformate is derived from diols of from 2 to 8 carbon atoms and polyether glycols of from 2 to 6 carbon atoms and from 2 to 8 repeat units.

25. The microcapsule of claim 23 wherein the chloroformate is selected from ethylene bis(chloroformate), 1,4-butanediol di(chloroformate), 1,6-hexanediol bis chloroformate, tri(ethylene glycol) bis(chloroformate), diethylene glycol bis-chloroformate, diglycolyl chloride, 1,4-phenylene bis(chloroformate), bisphenol A bis(chloroformate), bisphenol Z bis(chloroformate),

26. The microcapsule of claim 1 wherein the chloro component is one or more di- and/or or poly-sulfonyl chlorides.

27. The microcapsule of claim 26 wherein the sulfonyl chloride is selected from 1,3-benzenedisulfonyl chloride, 1,4-benzenedisulfonyl chloride, 1,3,5-benzenetrisulfonyl chloride, 4,4′-biphenyldisulfonyl chloride, 4,4′-methylenebis(benzenesulfonyl chloride), 4,4′-bis(chlorosulfonyl)diphenyl ether, 1,5-naphthalenedisulfonyl chloride, and 2,7-naphthalenedisulfonyl chloride.

28. A method of forming microcapsules comprising forming 1) an emulsion or dispersion of water phase composition and an oil phase composition, the water phase composition comprising (A) one or more protein-based components selected from proteins, peptides, polypeptides and combinations of any two or more of the foregoing and the oil phase composition comprising (B) the combination of i) an isocyanate component selected from the group consisting of one or more diisocyanates and/or poly-isocyanates and ii) a chloro component selected from the group consisting of a) di- and/or poly-acid chlorides, b) bis- and/or poly-chloroformates and/or c) di- and/or poly-sulfonyl chlorides; wherein (I) the weight ratio of the protein-based components (A) to the combination (B) is from 100:1 to 1:100 ((A):(B)) and (II) the weight ratio of the isocyanate component (i) to the chloro component (ii) is from 100:1 to 100:50 ((i):(ii)), and 2) subjecting the emulsion to conditions suitable for effecting the polymerization and cross-linking of the protein with the isocyanate and chloro components and allowing the reaction to continue for a sufficient time to effect the formation of the microcapsules.

29. The process of claim 28 wherein the process is an oil-in-water microencapsulation process and the oil phase further comprises a core material.

30. An article of manufacture incorporating the microcapsules according to claim 1.

31. The article of manufacture according to claim 30, wherein the article is selected from the group consisting of a soap, a surface cleaner, a laundry detergent, a fabric softener, a shampoo, a textile, a paper towel, an adhesive, a wipe, a diaper, a feminine hygiene product, a facial tissue, a pharmaceutical, a napkin, a deodorant, a heat sink, a foam, a pillow, a mattress, bedding, a cushion, a cosmetic, a medical device, packaging, an agricultural product, a cooling fluid, a wallboard, and insulation.
